(19) United States
(12) Patent Application Publication
(10) Pub. No.: US 2014/0120088 A1
(43) Pub. Date: May 1, 2014

(54) AGENTS FOR THE TREATMENT OF TUMORS

(71) Applicant: Antoine Carpentier, Paris (FR)

(72) Inventor: Antoine Carpentier, Paris (FR)

(73) Assignee: Assistance Publique-Hopitaux de Paris, Paris (FR)

(21) Appl. No.: 14/119,657

(22) PCT Filed: May 24, 2012
(86) PCT No.: PCT/EP2012/059744

§ 371 Date: Jan. 14, 2014

(30) Foreign Application Priority Data
May 24, 2011 (FR) 11/54531

(51) Int. Cl. A61K 39/395; A61K 31/7052

(52) U.S. Cl. CPC A61K 39/3955; A61K 31/7052
USPC 424/133.1; 514/44 R; 424/158.1

(57) ABSTRACT

The invention relates to the treatment of patients with a brain tumor or neoplastic meningitis, by the use of an agonist of the TLR9 receptor in combination with an anti-angiogenic product.

The invention relates to the treatment of patients with a brain tumor or tumor meningitis (neoplastic meningitis), by the use of an agonist of the TLR9 receptor in combination with an anti-angiogenic product.

Over the past years, clinical trials in the field of cancer immunotherapy have multiplied. Although the approaches have sometimes shown to be effective in terms of inducing an immune response in patients, the proportion of patients developing an objective clinical response has itself remained very low.

Thus, an approach of immunotherapy in patients with recurrent glioblastoma by local injection of oligodeoxynucleotides containing at least one dinucleotide CpG (Cytosine-phosphorothioate-Guanine) (CpG-ODN) (Carpentier et al., Neuro Oncol. 2010 April; 12(4):401-8) has been tested. This study did not achieve the expected benefit in terms of patient survival, although some patients have presented a long-term survival.

CpG-ODN are synthetic short single-stranded DNA molecules containing a cytosine <<C>> followed by a guanine <<G>>. The <<p>> refers to the phosphodiester backbone of DNA, but most CpG have a phosphorothioate backbone. When these CpG motifs are not methylated, they have an immunostimulatory effect (Weiner et al., 1997, Proc. Natl Acad Sci USA 94 (20): 10833-7).

These CpG-ODN are also described as immunostimulatory oligonucleotides in the literature.

CpG-ODN are agonists of the TLR9 receptor (Toll-like Receptor 9, also known as CD289. OMIM: 605474; MGI: 1932389; HomoloGene: 68126) which is expressed by dendritic cells and B cells (Rothenfusser et al., 2002 Human immunology 63 (12): 1111-9).

The activation of TLR9 receptors of dendritic cells by the CpG-ODN leads to the activation of these cells and to the production of cytokines such as interleukin (IL) 12 or type I interferons (IFN) (such as IFN-α or IFN-β).

The administration of CpG-ODN in or near a tumor can induce tumor rejection in multiple animal models (Carpentier et al., Clinical Cancer Research, 2000 6: 2469-73; Carpentier et al., Front Biosci. 2003 8: E115-27). In humans, several clinical trials have been conducted in glioblastomas with this molecule, which showed good tolerance, but still insufficient efficacy (Carpentier et al., Neuro-oncology, 2006 8: 60-2006; Carpentier et al., 2010 op. cit.).

VEGF (Vascular Endothelial Growth Factor) is a pro-angiogenic agent that plays a key role in tumor growth. A monoclonal antibody directed against VEGF, Bevacizumab, marketed by Roche under the name of Avastin®, already obtained marketing authorization in several cancer indications, such as colon cancer, lung cancer and glioblastoma recurrence.

A phase III trial using this antibody in the indication against de novo glioblastoma in combination with the standard treatment of this disease (radiotherapy and temozolomide) is currently underway. There is no recognized indication in neoplastic meningitis.

Neoplastic meningitis result from the invasion of intraventricular and meningeal spaces (subarachnoid space) by tumor cells (from a brain tumor or corresponding to meningeal metastasis of a systemic cancer accompanied or not by brain locations). The diagnosis is based on a lumbar puncture, and the use of MRI that can highlight tumor nodules or abnormal contrast shots, especially at the spinal cord level.

The prognosis of neoplastic meningitis secondary to solid tumors is catastrophic. The majority of patients have a median spontaneous survival of approximately 4 months (between 3 and 7 months), but this figure varies depending on the type of primary cancer (breast cancer: 7 months, small cell lung cancer: 4 months, melanoma: 3.6 months, malignant gliomas: 3 months). No treatment is effective (with the exception of methotrexate and Depocyte™ (Cytarabine) in hematological cancers).

Thus, there is no treatment to date for most neoplastic meningitis, that could offer relief to patients and/or increase their survival (Recht et Phuphanich Expert Rev Neurother 2004 July; 4(4 Suppl): SI 1-7; Glantz et al, Olin Cancer Res. 1999 November; 5(11):3394-402; Siegal J Neurooncol 1998 June-July; 38(2-3): 15-17; Chamberlain. Neurosurgery. 2003, 52:324-29).

Peak et al. (“Role of bevacizumab therapy in the management of glioblastoma”, Cancer Management and Research, 2010) disclose the use of anti-VEGF antibodies for treating glioblastomas. Different associations of anti-VEGF antibodies with irinotecan, temozolomide, chemotherapy and anticoagulants are discussed, as well as their interest.

Carpentier et al (“Intracerebral administration of CpG oligonucleotide for patients with recurrent glioblastoma: a phase II study.”, Neuro-Oncology, 2010) discloses the use of CpG ODN for treating brain tumors.

Chamberlain et al (“Emerging clinical principles on the use of bevacizumab for the treatment of malignant gliomas”. Cancer, 2010) discloses the use of anti-VEGF antibodies for treating glioblastomas. Associations discussed in Peak et al (op. cit.) are also mentioned. Others are suggested or tested (erlotinib, etoposide, fotemustine, carbamazepine). The use of bevacizumab for treating a neoplastic meningitis is suggested.

Carpentier et al (“Cancer immunotherapy with CpG-ODN”, Médecine Sciences, 2005) discusses the interest of immunostimulatory oligonucleotides in cancer immunotherapy. The combination of CpG ODN with antigens, antibodies or dendritic cells is reported to be effective. Discussed associations are OVA (melanoma), Herceptin® (breast cancer) and Rituxan® (non-Hodgkin lymphoma). The use of CpG for the treatment of brain tumor is discussed.

Pinhal-Enfield et al (“An angiogenic switch in macrophages involving synergy between Toll-like receptors 2, 4, 7, and 9 and adenosine A(2A) receptors.” The American Journal of Pathology, 2003) shows that TLR2, TLR4, TLR7 and TLR9 agonists increase the expression of VEGF in murine macrophages, inducing a change in their phenotype from inflammation to angiogenesis. Although such a mechanism may be of interest in the treatment of some diseases, brain tumor and neoplastic meningitis are not cited. This document suggests that agonists of TLR9 induce angiogenesis.

Zhao et al (“Carbon Nanotubes Enhance CpG Uptake and Potentiate Antiglioma Immunity”, Clinical Cancer Research, 15 février 2011) describes a study of the administration of CpG in the brain using carbon nanotubes (abstract).

El Andaloussi et al (“Stimulation of TLR9 with CpG ODN enhances apoptosis of glioma and probongs the survival of mite with experimental brain tumors”. Glia, 2006) discloses the beneficial effect of CpG in an experimental model of brain tumor.

Sorrentino et al (Int J Cancer. 2011 Jun. 15;128 (12):2815-22) show that the administration of CpG ODN increases the production of VEGF in a mouse model of lung carcinoma. This result is comparable to those reported by Pinhal-Enfield et al (op. cit.).

None of these documents discloses nor suggests the use of an association of a TLR9 receptor agonist and an anti-angiogenic agent for the treatment of a brain tumor or a neoplastic meningitis, although many discuss the interest a combination of different drugs for the treatment of brain tumor. In particular, none of these documents makes it possible to envisage the unexpected effect reported in the present application, namely synergy or cooperation between a TLR9 receptor agonist and an anti-angiogenic agent for the treatment of a brain tumor or neoplastic meningitis.

The results reported this application show that the use of an agent that is an agonist of the TLR-9 and of an anti-angiogenic agent improves survival of patients with neoplastic meningitis, and that it can control the evolution of a tumor in a relevant animal model.

Damiano et al (Proc. Natl. Acad. Sci. USA 2007, 104(30), 12468-12473) describe the combined use of a CpG-ODN and of bevacizumab in an animal model (nude mice) on a colon tumor xenograft. However, CpG-ODN were injected intraperitoneally, which does not correspond to an appropriate clinical use. In addition, phosphorothioate ODN not cross blood-brain barrier (Agrawal, 1991), which does not make it possible to extrapolate these results to intracerebrally localized tumors. Finally, the expression of TLR9 in humans is restricted mainly to B cells and plasmacytoid dendritic cells (pDCs), whereas the expression is broader in mice including the myeloid lineage (macrophages, monocytes, myeloid dendritic cells) (Carpentier, 2003).

Thus the results obtained in mice can't be systematically generalized in humans. There are also many examples of effective animal models combining CpG-ODN with other molecules (e.g. Damiano et al., (Clin Cancer Res 2006;12:577-583) who describe the combined use of CpG -ODN with cetuximab or gefitinib in xenograft models of colon cancer in nude mice) or the combination of bevacizumab with other molecules, whose results could not been confirmed in humans. Another example, among others, is the combination [TLR9 agonist+chemotherapy by dacarbazine (DTIC)], promising in a model of melanoma in animals (Najar and Dutz, 2008) but without efficacy in a randomized trial humans (Weber et al, 2009).

WO 2004/103301 also mentions that one could use CpG-ODN with bevacizumab, but doesn't provide any example of combination with this antibody. In addition, none of the reported results were so in humans.

Thus the results of the prior art obtained in animals can't be generalized in humans, or for intracerebral tumors. The present application reports, for the first time and surprisingly, the effectiveness of the CpG-ODN+anti-VEGF combination both in humans, in a clinical trial, and in an animal model

The invention thus relates to a product containing at least one TLR9 receptor (Toll-like Receptor 9) agonist agent in combination with an anti-angiogenic agent for its simultaneous, separate or sequential therapeutic use in the treatment of a patient with a tumor. This tumor is preferably a neoplastic meningitis or intracerebral tumor. This patient is a human.

In a particular embodiment said TLR9 receptor agonist agent is an oligonucleotide, and in particular a CpG-ODN, oligodeoxynucleotide cytosine-phosphorothioate-guanine, containing non-methylated cytosines and guanines.

This TLR9 agonist oligonucleotide, whether “CpG-ODN” or not (some may have patterns that are not CpG motifs as such, but which include changes in CpG motifs) is therefore an oligonucleotide that can be described as an immunostimulant.

As seen above, the immunostimulatory properties of nucleic acids, and CpG-ODN in particular are usually in relation with unmethylated 5′-CG, patterns which are under-represented in the DNA of the vertebrate (Krieg A M et al. Nature 1995; 374: 546-549).

Various patent applications or patents describe such oligonucleotides: one can cite US 20040006034, US 20030212029, U.S. Pat. No. 6,653,292, U.S. Pat. No. 6,239,116, U.S. Pat. No. 6,207,646, U.S. Pat. No. 6,194,388, U.S. Pat. No. 6,429,199, U.S. Pat. No. 6,406,705, U.S. Pat. No. 6,218,371, U.S. Pat. No. 6,214,806, U.S. Pat. Nos. 6,218,371, 6,727,230, WO 01/51500, WO 03/035695, EP 1162982.

Thus, synthetic oligodeoxynucleotides (ODN) containing such motifs (CpG-ODN) retain marked immunostimulatory properties, especially the phosphorothioate ODN that are resistant to nucleases. CpG-ODN are internalized by cells through endocytosis and bind to TLR9 in the endosomes. In humans, TLR9 are predominantly selectively by B cells and plasmacytoid dendritic cells (pDCs).

The B cell activation by CpG-ODN results in secretion of cytokines such as IL-6 or IL-10, cellular proliferation, inhibition of apoptosis induced by various agents and immunoglobulin secretion (Klinman, Nat Rev Immunol 2004; 4: 249-59; Krieg, Curr Oncol Rep 2004; 6: 88-95; Carpentier et al, Front Biosci 2003; 8: El 15-27).

Activation of human plasmacytoid dendritic cells causes their maturation, secretion of many cytokines such as TNFα, alpha or gamma interferon. IL-6 or IL-12 and expression of co-stimulatory molecules (CD40, CD80, CD86) and of CCR7, a receptor which controls migration to the T areas of the lymph node. The secretion of IL-12 and IFN gamma directs the immune response to the Th1 profile, and can even turn a Th2 response to a Th1 one.

The biological activity of an oligonucleotide depends on many variables, still imperfectly known, such as, for example, chemical modifications of the backbone, the nucleotide sequence surrounding the CpG motif or the number of CpG motifs. Some immunostimulatory oligonucleotides without CpG motifs have thus been described (US 20040006032).

The chemical modification of the backbone, which is naturally formed in DNA by phophodiesters bonds (sensitive to nucleases), plays an important role both in improving the stability of the ODN but also by changing its immunostimulatory properties. Several types of stabilized oligonucleotides have been created (Iyer (1999) Curr Opinion Mol Therap 1; 344-358). The most frequently used CpG-ODN are the phosphorothioate oligodeoxynucleotides, or mixed phosphorothioate/phosphodiester oligodeoxynucleotides (e.g. phosphorothioate ends/phosphodiester center; or phosphorothioate ODNs, except for cytosine-guanosine bonds which are phosphodiester). The synthesized oligodeoxynucleotides can be purified according to their stereoisomerism to change or improve their biological activity. The immunostimulating activity of highly modified synthetic oligonucleotides, due to the presence of unnatural purines or pyrimidines bases (US 20030181406; US 20020137714; U.S. Pat. No. 6,562,798; US 20030186912), of chimeric DNA/RNA molecules or of ODN-linker-ODN (US 20040052763; US 20030225016; US 20030199466; US 20030175731; Wagner, Curr Opin Immunol. 2008 August;20(4):396-400; Struthers et al., Cell Immunol. 2010 Mar. 10) or the addition of non-oligonucleotide ligands has also been reported (reviewed by Uhlmann et Vollmer, Curr. Opin. Drug Discov. Devel., 2003, 6, 204-217).

Several families of CpG-ODN have already been characterized. The more classic, referred to as “type B” (or “K”), is characterized by a strong activation of B cells and dendritic cells. The “type A” (or “D”) ODN are characterized by a strong activation of NK and secretion of alpha-interferon by pDCs. The type C ODN combine the properties of the previous two types. Reviews on this matter have been published (Klinman, Nat Rev Immunol 2004; 4: 249-59; Krieg Curr Oncol Rep 2004; 6: 88-95).

A new class of CpG-ODNs, called class P has recently been described (Samulowitz et al., Oligonucleotides. 2010 April; 20(2):93-101). These oligonucleotides contain two palindromic sequences and can thus form multimeric units. The effectiveness of these P molecules (especially regarding the ability to induce the expression of type I interferons) is greater than that of type C CpG-ODN.

In a specific embodiment, a CpG-ODN of sequence SEQ ID No 1, 5′-TAAACGTTATAACGTTATGACGTCAT-3′ in which the backbone is phosphorothioate, is used.

Angiogenesis is a process which describes the growth of new blood vessels (neovascularization) from pre-existing vessels.

This process is controlled by various activators and inhibitors produced by the healthy and tumor cells. Activators include pro-angiogenic molecules (VEGF, FGF (Fibroblast Growth Factor), PDGF (Platelet-Derived Growth Factor)) and inhibitors, anti-angiogenic molecules (angiostatin, thrombospondin).

By anti-angiogenic agent, it is meant any endogenous or exogenous agent capable of inhibiting angiogenesis. In particular, one can cite any inhibitor of VEGF, FGF and PDGF.

A number of inhibitors of angiogenesis are known. As an illustration, one can cite a review on these products (Samant et Shevde Oncotarget. 2011 March;2(3):122-34: Recent Advances in Anti-Angiogenic Therapy of Cancer).

This anti-angiogenic agent is preferably an inhibitor of VEGF, i.e. an agent limiting or inhibiting the action of endogenous VEGF. The agent inhibiting VEGF may be an antibody (such as bevacizumab, monoclonal antibody) or a small organic molecule (such as sorafenib or sunitinib, which inhibit VEGF receptors but also other receptors). Mention may also be made of other types of agents, such as aflibercept, a fusion protein developed by Sanofi Aventis, which “traps” VEGF or prolactin.

The VEGF inhibitor is preferably a monoclonal antibody.

In a preferred embodiment, said tumor is a primary benign or malignant brain tumor, such as a meningioma, a glial tumor (glioma), a medulloblastoma, or a tumor of the pineal region.

In this particular embodiment, the tumor is in particular an anaplastic glioma, such as an anaplastic astrocytoma, an anaplastic oligodendroglioma, an anaplastic mixt glioma, or a glioblastoma.

In another embodiment, said tumor is a brain metastastic extension of an extra-cerebral cancer, such as a lung cancer, a breast cancer, a digestive tract cancer, a melanoma or a gynecological cancer.

In another preferred embodiment, said tumor is a neoplastic meningitis secondary to a primary brain tumor, or a meningeal metastasis of an extra-cerebral cancer.

In particular, said neoplastic meningitis is secondary to a benign or malignant primary brain tumor, selected from a meningioma, a glial tumor (glioma), a medulloblastoma, a tumor of the pineal region, an anaplastic astrocytoma, an anaplastic oligodendroglioma, an anaplastic mixt glioma, and a glioblastoma.

Said neoplastic meningitis may also be secondary to subarachnoid metastasis of a lung cancer, a breast cancer, a digestive tract cancer (in particular a cancer of the colon or of the stomach), a melanoma or a gynecological cancer.

In another embodiment, said tumor is a lung cancer (including small cell lung cancer), a breast cancer, a digestive tract cancer (including a cancer of the colon or stomach), a melanoma or a gynecological cancer.

In a particular embodiment, the agents making-up the combination are administered to the patient following completion of anti-tumor therapy, especially when the antitumor treatment is surgical resection or radiotherapy, administration of anti-cancer substances such as chemotherapy, immunotherapy or any other form of treatment (ultrasonic, electromagnetic fields), or a combination of these treatments.

It is also possible to perform the administration of the combination of agents according to the invention concomitantly with the administration of other antitumor agents, or before another antitumor treatment, as mentioned above.

The invention also relates to a method of treating a patient having a tumor (as defined above) comprising the step of administering to said patient a TLR9 receptor agonist agent in combination with an anti-angiogenic agent.

The administration of these compounds is not necessarily concomitant.

In a preferred embodiment, the TLR9 receptor agonist agent is administered at a frequency ranging from once a week to once every two months, preferably between once a week and once a month, more preferably once every two weeks. A dose of between 0.5 to 40 mg, preferably between 10 and 20 mg of oligonucleotide is used. This agent is preferably administered intrathecally for brain tumors and neoplastic meningitis and/or subcutaneously. For other types of tumors, this agent is preferably administered locally (including directly into the tumor for solid tumors, or adjacent to the tumor), in order to induce immunostimulation in the vicinity of the tumor. It is therefore in a form suitable for such intrathecal and/or subcutaneously administration, or in a form suitable for intravenous administration.

The anti-angiogenic agent is administered according to the recommendations of each molecule. Thus, bevacizumab is administered at a frequency between once a week and once every two months, preferably one every two weeks. The dose is determined by the practitioner, and is generally less than 15 mg/kg, more particularly of 10 mg/kg. In a preferred embodiment, the anti-angiogenic agent is administered intravenously.

### DESCRIPTION OF THE FIGURES

FIG. 1: Effect of an anti-VEGF vaccination combined with a local immunotherapy on tumor growth in a model of syngeneic subcutaneous glioma in rats. White circles: control; white squares: anti-VEGF immunization; black circles: CpG-ODN; black squares: anti-VEGF immunization+CpG-ODN.

FIG. 2: Effect of an anti-VEGF vaccination combined with a local immunotherapy on animal survival in a model of syngeneic subcutaneous glioma in rats. (the animals were sacrificed when tumors reached 30 mm in diameter). White circles: control; white squares: anti-VEGF immunization; black circles: CpG-ODN; black squares: anti-VEGF immunization+CpG-ODN.

FIG. 3: evolution of brain MRI in a patient during the course of treatment. The treatment, administered between each MRI, (compound used and number of injections) is specified.

### Examples

In all examples, the used CpG-ODN have the sequence SEQ ID No. 1, with a phosphorothioate backbone.

### Example 1

Rats were immunized against VEGF-A using a recombinant VEGF protein and an immune adjuvant (polyarginine). The administration of VEGF thus induces an immune response that ultimately leads to an anti-angiogenic effect.

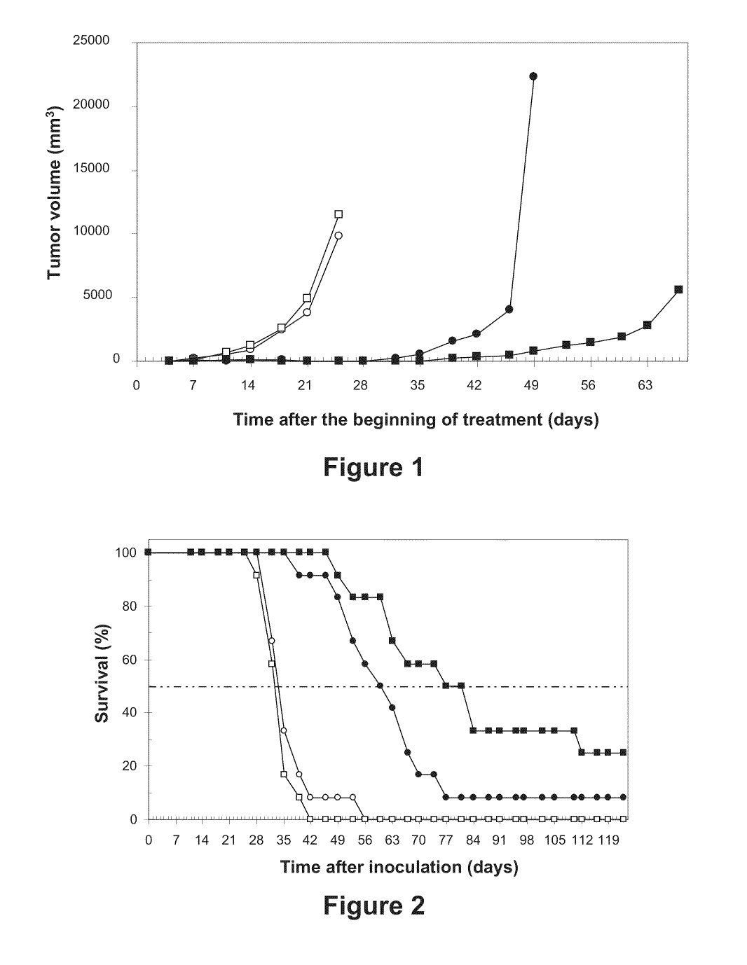

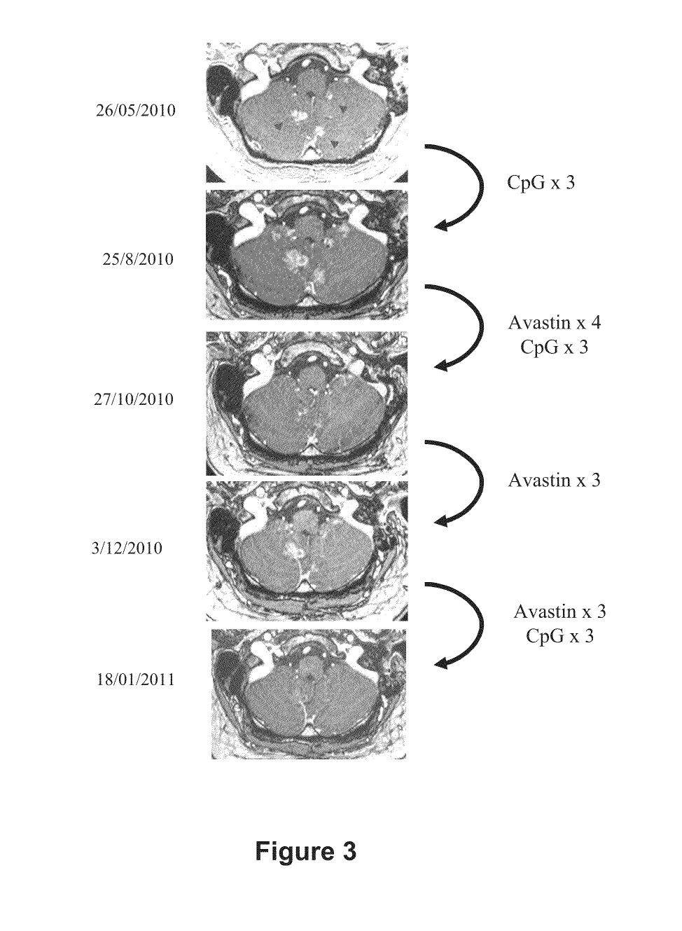

FIGS. 1 and 2 show that in a model of syngeneic glioma (RG2—Fischer Rat), the anti-VEGF vaccine acts in synergy with a local immunotherapy treatment (peri-tumoral injections of CpG-ODN) to inhibit tumor growth and increase the survival of animals. Treatment (vaccination and/or local immunotherapy) was administered 7 days after the RG2 tumor implantation and repeated 14 days later (n=12 animals per group).

Protocol

Animals: Fisher female rats aged 5 weeks

At days −5 or −7, sub-cutaneous inoculation of 100,000 cells of rat glioma (RG2) (left flank).

At days 0 and 14, subcutaneous distal injection of the anti-VEGF vaccine (hVEGF-A+ adjuvant) and peritumoral injection of CpG-ODN.

TABLE I
Summary of the protocol applied to mice

| | subcutaneous injection | subcutaneous injection | |
|---|---|---|---|
| | (right flank) | (left flank) | Date |
| Group | — | — | D 0, D 14 |
| #1 | | | |
| Group | — | CpG-ODN | D 0, D 14 |
| #2 | | | |
| Group | Human VEGF + adjuvant | — | D 0, D 14 |
| #3 | | | |
| Group | Human VEGF + adjuvant | CpG-ODN | D 0, D 14 |
| #4 | | | |

Tumor growth was measured (twice a week) and the survival of animals was observed (euthanasia when the tumor diameter exceeded 30 mm).

Thus, the combination of an injected product inducing an immunity against VEGF (thus ultimately inducing an anti-angiogenic effect) and a TLR9 agonist allows control of the tumor in this murine model.

### Example 2

Four patients had high-grade glioma with subarachnoid extension. The treatment protocol of patients was as follows:

Subcutaneous injection of 0.3 mg/kg of CpG-ODN at D0, D7, D14, D21 et D28 (capped at 25 mg per injection, for patients>83 kg)

Intrathecal administration of CpG-ODN (7 mg) (in the cerebrospinal fluid) three times, with a two-week interval between each injection (D0, D14 and D28). (In 2 patients, the intrathecal injections were continued every 2 to 4 weeks for several months). (Other intrathecal injections of larger amounts of CpG-ODN (14 mg or 17 mg), again in three steps with a two weeks interval between each injection are currently tested, in other patients in a clinical trial.)

Intravenous perfusions of Avastin from 5 to 10 mg/kg every 2 weeks, started, depending on the cases, before, during or after the beginning of CpG-ODN.

Of these 4 patients, 2 showed a remarkable development: while the median survival in such patients is about 3 months, they presented clinical and radiological improvement, and were almost asymptomatic, 6 and 7 months respectively, after the beginning of the combined treatment. These patients had received different series of three injections of CpG according to the protocol described above (7 mg of CpG injection).

This effect can't be attributed to CpG alone, these two patients having presented only a slight improvement with CpG, before the combined treatment with bevacizumab. Bevacizumab could in theory be the origin of such stabilization, but one of the patients showed a progression under bevacizumab alone, then a response when intrathecal CpG was resumed (FIG. 2). Thus, in at least this patient, the combination of CpG and Avastin seems to be the cause of this efficiency.

### REFERENCES

Carpentier et al. (2000). Successful treatment of intracranial gliomas in rats with oligodeosynucleotides containing CpG motifs. Clinical Cancer Research, 6: 2469-73.

Carpentier et al. (2003). CpG-oligonucleotides for cancer immunotherapy: review of the literature and potential applications in malignant glioma. Front Biosci. 8: E115-27.

Carpentier et al. (2006). Phase I trial of a CpG oligodeoxynucleotide for patients with recurrent glioblastoma. Neuro-oncology, 8: 60-6.

Carpentier et al., Neuro Oncol. 2010 April;12(4):401-8)

Chamberlain. Neurosurgery. 2003, 52:324-29

Damiano et al Clin Cancer Res 2006;12:577-583. Novel Toll-Like Receptor 9 Agonist Induces Epidermal Growth Factor Receptor (EGFR) Inhibition and Synergistic Antitumor Activity with EGFR Inhibitors

Damiano et al. Proc. Natl. Acad. Sci. USA 2007, 104(30), 12468-12473

Glantz. A randomized controlled trial comparing intrathecal sustained-release cytarabine (DepoCyt) to intrathecal methotrexate in patients with neoplastic meningitis from solid tumors. Clin Cancer Res. I 999 November; 5(11):3394-402.

Iyer et al. Nucleotide analogs as novel anti-hepatitis B virus agents. Curr Opin Pharmacol. 2005 October;5(5):520-8. Review

Klinman. Immunotherapeutic uses of CpG oligodeoxynucleotides. Nat Rev Immunol. 2004 April;4(4):249-58. Review. No abstract available.

Krieg et al. CpG motifs in bacterial DNA trigger direct B-cell activation, Nature. 1995 Apr. 6;374(6522):546-9.

Krieg. Antitumor applications of stimulating toll-like receptor 9 with CpG oligodeoxynucleotides. Curr Oncol Rep. 2004 March;6(2):88-95.

Recht et Phuphanich: Treatment of neoplastic meningitis: what is the standard of care? Expert Rev Neurother 2004 July; 4(4 Suppl): SI 1-7.

Rothenfusser et al., 2002 Human immunology 63 (12): 1111-9.

Samulowitz et al., Oligonucleotides. 2010 April;20(2):93-101 A novel class of immune-stimulatory CpG oligodeoxynucleotides unifies high potency in type I interferon induction with preferred structural properties.

Siegal: Leptomeningeal metastases: rationale for systemic chemotherapy or what is the role of intra-CSF-chemotherapy? J Neurooncol I 998 June-July;38(2-3): I5 1-7.

Struthers et al. Synthesis and immunological activities of novel agonists of toll-like receptor 9. Cell Immunol. 2010 Mar. 10.

Uhlmann et Vollmer, Curr. Opin. Drug Discov. Devel., 2003, 6, 204-217). Recent advances in the development of immunostimulatory oligonucleotides.

Wagner. The sweetness of the DNA backbone drives Toll-like receptor 9. Curr Opin Immunol. 2008 August;20(4):396-400

Weiner et al., 1997, Proc. Natl Acad Sci USA 94 (20): 10833-7)

1-17. (canceled)

18. A method for treating a patient with a brain tumor or neoplastic meningitis, comprising the steps of administering to said patient at least one TLR9 receptor (Toll-like Receptor 9) agonist agent and an anti-angiogenic agent.

19. The method of claim 18, characterized in that said TLR9 receptor agonist agent is an oligonucleotide.

20. The method of claim 19, wherein said oligonucleotide is a cytosine-phosphorothioate-guanine oligodeoxynucleotide containing non-methylated cytosines and guanines.

21. The method of claim 19, wherein said oligonucleotide has a phosphorothioate backbone.

22. The method of claim 19, wherein said oligonucleotide has the sequence SEQ ID No 1.

23. The method of claim 18, wherein said anti-angiogenic agent is an inhibitor of VEGF (Vascular Endothelial Growth Factor).

24. The method of claim 23, wherein said agent inhibitor of VEGF is an antibody.

25. The method of claim 24, wherein said antibody is bevacizumab.

26. The method of claim 18, wherein said tumor is a glioma or a glioblastoma.

27. The method of claim 18, wherein said tumor is a brain metastastic extension of an extra-cerebral cancer, such as a lung cancer, a breast cancer, a digestive tract cancer, a melanoma or a gynecological cancer.

28. The method of claim 18, wherein said tumor is a neoplastic meningitis secondary to a primary brain tumor, or a meningeal metastasis of an extra-cerebral cancer.

29. The method of claim 28, wherein said neoplastic meningitis is secondary to a benign or malignant primary brain tumor, selected from a meningioma, a glial tumor (glioma), a medulloblastoma, a tumor of the pineal region, an anaplastic astrocytoma, an anaplastic oligodendroglioma, an anaplastic mixt glioma, and a glioblastoma.

30. The method of claim 28, wherein said neoplastic meningitis is secondary to subarachnoid metastasis of a lung cancer, a breast cancer, a digestive tract cancer (in particular a cancer of the colon or of the stomach), a melanoma or a gynecological cancer.

31. The method of claim 18, wherein said TLR9 receptor agonist agent is administered intrathecally and/or subcutaneously.

32. The method of claim 18, wherein said anti-angiogenic agent is administered intravenously.

33. The method of claim 18, wherein said TLR9 receptor agonist agent is administered intrathecally at a frequency ranging between once a week and once every two months.

34. The method of claim 18, wherein said TLR9 receptor agonist agent is administered at a dose between 0.5 and 40 mg.

35. The method of claim 18, wherein said TLR9 receptor agonist agent and said anti-angiogenic agent are administered simultaneously.

36. The method of claim 18, wherein said TLR9 receptor agonist agent and said anti-angiogenic agent are administered separately.

37. The method of claim 18, wherein said TLR9 receptor agonist agent and said anti-angiogenic agent are administered sequentially.


SEQUENCE LISTING (1 sequence)

SEQ ID NO: 1 - DNA, 26 bp, Artificial
  Synthetic oligonucleotide CpG
taaacgttataacgttatgacgtcat